(19) United States
(12) Patent Application Publication
(10) Pub. No.: US 2005/0226813 A1
(43) Pub. Date: Oct. 13, 2005

(54) Labelled somatostatin analogs backbone cyclized through metal complexation

(76) Inventors: Thomas A. Bonasera, Cambridge (GB); Gil Fridkin, Tel Aviv (IL); Chaim Gilon, Jerusalem (IL)

Correspondence Address: WINSTON & STRAWN LLP, 1700 K STREET, N.W., WASHINGTON, DC, 20006, US

(21) Appl. No.: 11/021,885

(22) Filed: Dec. 23, 2004

Related U.S. Application Data
(63) Continuation of application No. PCT/IL03/00531, filed on Jun. 24, 2003.

(30) Foreign Application Priority Data
Jun. 24, 2002 (IL) IL150384

(51) Int. Cl. A61K 51/00; C07K 14/655

(52) U.S. Cl. 424/1.69; 514/9; 530/311

(57) ABSTRACT

Novel diagnostic and therapeutic peptides disclosed herein are somatostatin analogs backbone cyclized through metal complexation, and having improved somatostatin receptor subtype affinity and selectivity. These backbone cyclized peptide analogs possess unique and superior properties over other analogs, including chemical and metabolic stability, selectivity, increased bioavailability and improved pharmacokinetics. Pharmaceutical compositions that include these backbone cyclized somatostatin analogs, radiolabelled analogs, reagents for synthesizing same, and methods of using such compositions for diagnostic and therapeutic purposes are also disclosed.

### CROSS-REFERENCE TO RELATED APPLICATIONS

This application is a continuation of International Application PCT/IL2003/00531 filed Jun. 24, 2003, the entire content of which is expressly incorporated herein by reference.

### FIELD OF THE INVENTION

The present invention relates to Na backbone cyclic labelled somatostatin peptide analogs which are cyclized through complexation with metal, to pharmaceutical compositions containing same, to reagents for synthesizing same, and to methods for using such compounds for diagnosis and therapy.

### BACKGROUND OF THE INVENTION

Somatostatin (SST) is a cyclic tetradecapeptide found both in the central nervous system and in peripheral tissues. It was originally isolated from mammalian hypothalamus and identified as an important inhibitor of growth hormone secretion from the anterior pituitary. Its multiple biological activities include inhibition of the secretion of glucagon and insulin from the pancreas, regulation of most gut hormones and regulation of the release of other neurotransmitters involved in motor activity and cognitive processes throughout the central nervous system (for review see Lamberts, Endocrine Rev., 9: 427, 1988).

The diverse physiological effects of SST are induced by selective and high affinity binding to receptors that are members of the seven transmembrane segment receptor superfamily (reviewed in Reisine T., Bell G. I., Endocrinology Rev., 16: 427-442, 1995). So far, five SST receptor subtypes have been isolated and cloned designated SST-R1 through SST-R5. These receptors are characterized by a high degree of sequence homology, but are linked to different multiple cellular effector systems. The receptor subtypes recognize both naturally-occurring and synthetic ligands with different affinities.

In its natural form, SST has limited use as a therapeutic agent since it exhibits two undesirable properties: poor bioavailability and short duration of action. For these reasons, great efforts have been made to find SST analogs that will have superiority in potency, biostability, duration of action or selectivity.

### CROSS-REFERENCE TO RELATED APPLICATIONS

This application is a continuation of International Application PCT/IL2003/00531 filed Jun. 24, 2003, the entire content of which is expressly incorporated herein by reference.

### FIELD OF THE INVENTION

The present invention relates to Na backbone cyclic labelled somatostatin peptide analogs which are cyclized through complexation with metal, to pharmaceutical compositions containing same, to reagents for synthesizing same, and to methods for using such compounds for diagnosis and therapy.

### BACKGROUND OF THE INVENTION

Somatostatin (SST) is a cyclic tetradecapeptide found both in the central nervous system and in peripheral tissues. It was originally isolated from mammalian hypothalamus and identified as an important inhibitor of growth hormone secretion from the anterior pituitary. Its multiple biological activities include inhibition of the secretion of glucagon and insulin from the pancreas, regulation of most gut hormones and regulation of the release of other neurotransmitters involved in motor activity and cognitive processes throughout the central nervous system (for review see Lamberts, Endocrine Rev., 9: 427, 1988).

The diverse physiological effects of SST are induced by selective and high affinity binding to receptors that are members of the seven transmembrane segment receptor superfamily (reviewed in Reisine T., Bell G. I., Endocrinology Rev., 16: 427-442, 1995). So far, five SST receptor subtypes have been isolated and cloned designated SST-R1 through SST-R5. These receptors are characterized by a high degree of sequence homology, but are linked to different multiple cellular effector systems. The receptor subtypes recognize both naturally-occurring and synthetic ligands with different affinities.

In its natural form, SST has limited use as a therapeutic agent since it exhibits two undesirable properties: poor bioavailability and shbrt duration of action. For these reasons, great efforts have been made to find SST analogs that will have superiority in potency, biostability, duration of action or selectivity.

Somatostatin in Cancer

Because SST receptors are present in high density in many endocrine and non-endocrine tumors, diagnosis and treatment were attempted using radiolabelled SST analogs in cancer patients. Lost tumors express multiple SST receptor-subtypes, although the SST-R2 subtype is most predominantly expressed. Radiolabelled receptor-specific compounds can detect primary sites, identify occult metastatic lesions, guide surgical intervention, stage tumors, predict efficacy of certain therapeutic agents or, when labelled with suitable radionuclides, be useful radiotherapeutic agents. The abundance of high affinity SST receptors in various tumors (e.g. most endocrine-active tumors) enables the use of radiolabeled SST analogs for in vivo identification, visualization and localization of these tumors (Lamberts et al. N. Engl. J. Med., 334:246 1996). Binding studies and autoradiography using radiolabelled SST or its analogs, have shown that 80-90% of all neuroendocrine tumors of the gastrointestinal tract possess high numbers of SST receptors. It was demonstrated that carcinoid tumors possesses multiple SST receptor subtypes and that SST analogs such as Octreotide, which preferentially bind to receptor type 2 and 5, can be used in diagnosis and medical treatment of these tumors (Nilsson et al., Br J Cancer, 77:632, 1998). Based on binding studies of the cloned receptors, SST-R2 has been suggested to be the main target for Octreotide and a prerequisite for tumor imaging.

Radiolabelled Somatostatin Analogs as Diagnostic/Therapeutic Agents

Scintigraphy using labelled SST analog tracers helps to localize tumors and to evaluate the potential for chronic treatment of patients with inoperable SST receptor-positive tumors.

One method for using radiolabelled SST analogs is to label tyrosine containing analogs with iodine. International patent application WO 96/39161 discloses multi-tyrosinated SST analogs in which the N-terminal of the peptides is extended with tyrosine residues, for radioiodination and subsequent diagnosis and treatment.

One application of radiolabelled SST analogs is radio-guided surgery. Surgical intervention can be optimized by intraoperative detection of tissue-bound (¹²⁵I-Tyr3)-Octreotide administered before operation. This technique has been successfully utilized in surgery of medullary thyroid cancer, carcinoids and islet cell tumors. High specific activity is achieved by the multi-tyrosinated SST analogs as a result of multiple sites for iodination provided by the additional tyrosines. Another labeling method is reduction of a disulfide bridge, which provides two sulffiydryl groups for chelation with ^{99m}Tc (Kolan and Thakur Peptide Res., 9:144, 1996). Certain peptides can be labeled directly without a loss of functional specificity but others must be labeled using bifunctional chelating agents, which are covalently coupled to the analogs on one hand and form a complex with radiometals on the other hand. Methods for labeling peptides with ^{99m}Tc are described in U.S. Pat. No. 5,716,596 and U.S. Pat. No. 5,620,675. A series of patents on radiolabelled SST analogs, describes cyclic (U.S. Pat. No. 5,932,189, WO 95/00553 and WO 96/04308) and linear (U.S. Pat. No. 5,620,675, WO 95/03330) peptides with 10-16 residues and high affinity for SST receptors. The cyclic peptides disclosed do not comprise a disulfide bond. An N₂S₂ type chelating ligand containing two nitrogen and two sulfur atoms for chelate formation, and use for cyclic and linear hexapeptide SST analogs, is disclosed in international applications WO 96/11954 and WO 96/11918. A disulfide-bridged SST analog with specific chelating groups is claimed in European application no. 714911. Analogs that contain at least 2 cysteine residues that form a disulfide or wherein the disulfide is reduced to the sulfhydryl form are disclosed in U.S. Pat. No. 5,225,180. The compounds are stated to have improved tumor/kidney distribution ratios over conventional SST analogs, thus reducing kidney radiation exposure. International application WO 94/00489 and U.S. Pat. No. 5,871,711 disclose SST-derived peptide reagents for preparation of scintigraphic imaging agents. The SST analogs are labeled with ^{99m}Tc, ¹⁸⁶Re and ¹⁸⁸Re through complexation. U.S. Pat. No. 5,382,654 describes aminothiol ligands (N₂S₂ and N₃S) which can be conjugated to a SST analog peptide and can accommodate a metal ion, which can be a radiometal. For diagnostic purposes, ^{99m}Tc and ⁶²Cu are suggested for complex formation, while ¹⁸⁶Re, ⁶⁷Cu, ¹⁸⁸Re and ⁶⁰Co ions can be used for radiotherapy.

The effect of labeling methods and peptide sequence on ^{99m}Tc SST analogs was reviewed by Decristoforo C. and Mather S. J. (Eur. J. Nucl. Med., 26:869, 1999). It is concluded that the selection of the labeling approach as well as the right choice of the peptide structure are crucial for labeling peptides with ^{99m}Tc to achieve complexes with favorable activity and biodistribution. The authors further stated that advantages due to different receptor specificity remain a topic for further investigations.

A number of ^{99m}Tc-labeled bioactive peptides have proven to be useful diagnostic imaging agents. Pearson et al. (J. Med. Chem., 39:1361, 1996) describe the chemistry and biology of ^{99m}Tc labeled SST analogs.

A radiolabelled SST analog, ¹¹¹n-DTPA-(D)Phe-Octreotide (OctreoScan, Mallinkrodt), has high diagnostic capacity for neuroendocrine tumors and lymphomas while its applicability for other tumors such as melanomas is lower. Labeled Octreotide analogs bind to SST-R and SST-R5. Octreotide labelled with ¹¹¹In has been shown to detect a variety of neuroendocrine tumors with high specificity and sensitivity and becomes a valuable tool in diagnosis, but it suffers form at least one major drawback: the cost. Vapreotide (RC-160) was labeled with ^{99m}Tc directly and also by using a bifunctional chelating agent and was successfully evaluated in nude mice bearing experimental human prostate cancer. The compound ^{99m}Tc-Depreotide was successfully used in the evaluation of solitary pulmonary nodules in phase II/III clinical trial (Blum et al., Chest 117:1232, 2000). SST receptor imaging has been used successfully (utilizing ¹¹¹In-pentetreotide) for detection of cardiac allograft rejection (Aparici et al. Eur. J. Nuc. Med. 27:1754, 2000). Cardiac rejection process usually presents with lymphocyte infiltration, which indicates the severity of the rejection and the necessity of treatment. Activated lymphocytes express SST receptors thus SST receptor imaging could be used to target them. Somatostatin receptor imaging may predict impending rejection at least one week before the endomyocardial biopsy becomes positive and thus allow earlier intervention in the event of rejection.

A variety of radionuclides are known to be useful for radioimaging, including ⁶⁷Ga, ⁶⁸Ga, ^{99m}Tc, ¹¹¹In, or 1231. The sensitivity of imaging methods using radioactively-labeled peptides is much higher than other techniques known in the art, since the specific binding of the radioactive peptide concentrates the radioactive signal over the cells of interest, for example, tumor cells. This is particularly important for endocrine-active gastrointestinal tumors, which are usually small, slow-growing and difficult to detect by conventional methods. Technetium-99m (^{99m}Tc, t_{1/2}=6 h, E_{γ}=140 keV) is the radionuclide of choice by virtue of its cost-effectiveness, availability and desirable nuclear characteristics. It is a decay product of ⁹⁹Mo. Because of its short half-life, it does not induce unnecessary radiation burden to a patient long after examinations are carried out, and its gamma ray energy is highly efficient for external imaging. ^{99m}Tc is used in over 90% of the diagnostic nuclear medicine procedures. Other radionuclides have effective half-lives, that are much longer (for example, ¹¹¹In, which has a half-life of 60-70 h), are toxic (for example In with its auger electron emission) or are expensive (¹¹¹In which is a cyclotron-produced radionuclide).

U.S. Pat. No. 4,980,147 discloses ^{99m}Tc compounds used as radiopharmaceutical imaging agents and particularly for conducting renal function imaging procedures. The preferred compound claimed is ^{99m}Tc-mercaptoacetyl-glycylglycylglycine (^{99m}Tc-MAG3). This and related compounds are used without conjugation with a SST or other peptide analog. U.S. Pat. No. 4,883,862 discloses the compound mercaptosuccinyl-glycylglycylglycine and its complexes with ^{99m}Tc for use as renal agents. The mercaptosuccinyl-glycylgiycylglycine is made by coupiing glycylglycylglycine with S-acetyl-mercapto succinic anhydride.

WO01/02022 disclosed linear alpha melanlocyte stimulating hormone analogs cyclized through oxorhenium(V) and oxotechnetium(V), providing stable complexes able to reach their target in vivo. These novel compounds are candidates for diagnostic imaging and targeted radiopeptide therapy of melanotropin receptor-expressing melanoma.

Improved Peptide Analogs

As a result of major advances in organic chemistry and in molecular biology, many bioactive peptides can now be prepared in quantities sufficient for pharmacological and clinical use. Thus in the last few years new methods have been established for the treatment and diagnosis of illnesses in which peptides have been implicated. However, the use of peptides as therapeutic and diagnostic agents is limited by the following factors: a) tissue penetration; b) low metabolic stability towards proteolysis in the gastrointestinal tract and in serum; c) poor absorption after oral ingestion, in particular due to their relatively high molecular mass or the lack of specific transport systems or both; d) rapid excretion through the liver and kidneys; and e) undesired side effects in non-target organ systems, since peptide receptors can be widely distributed in an organism.

It would be desirable to achieve peptide analogs with greater specificity thereby achieving enhanced clinical selectivity. It would be most beneficial to produce conformationally constrained peptide analogs overcoming the drawbacks of the native peptide molecules, thereby providing improved therapeutic properties.

A novel conceptual approach to the conformational constraint of peptides was introduced by Gilon, et al., (Biopolymers 31:745, 1991) who proposed backbone to backbone cyclization of peptides. The theoretical advantages of this strategy include the ability to effect cyclization via the carbons or nitrogens of the peptide backbone without interfering with side chains that may be crucial for interaction with the specific receptor of a given peptide. Further disclosures by Gilon and coworkers (WO 95/33765, WO 97/09344, U.S. Pat. No. 5,723,575, U.S. Pat. No. 5,811,392, U.S. Pat. No. 5,883,293 and U.S. Pat. No. 6,265,375), provided methods for producing building units required in the synthesis of backbone cyclized peptide analogs. The successful use of these methods to produce backbone cyclized peptide analogs of bradykinin analogs (U.S. Pat. No. 5,874,529), and backbone cyclized peptide analogs having somatostatin activity was also disclosed (WO 98/04583, WO 99/65508, U.S. Pat. No. 5,770,687, U.S. Pat. No. 6,051,554 and U.S. Pat. No. 6,355,613). WO 02/062819 of one of the present inventors, discloses radiolabelled-backbone cyclized somatostatin analogs for diagnostic and therapeutic uses. All of these methods and analogs are incorporated herein in their entirety, by reference.

There remains a need for synthetic SST analogs having increased in vivo stability, to be used therapeutically, as scintigraphic agents when labelled with Tc-99m or other detectable isotopes for use in imaging tumors in vivo, and as radiotherapeutic agents when radiolabelled with a cytotoxic radioisotope such as rhenium-188. It would be desirable to achieve peptide analogs with greater affinity and specificity to receptor subtypes thereby achieving enhanced diagnostic selectivity to elucidate the specific SST receptor profile in each individual for planning further therapy and/or surgery. Backbone cyclized SST analogs that specifically fulfill these needs are provided by this invention.

None of the background art teaches or suggests the somatostatin analogs backbone cyclized via complexation with a metal, disclosed herein having improved diagnostic and therapeutic activity and selectivity.

### SUMMARY OF THE INVENTION

The present invention provides novel somatostatin analogs that are backbone cyclic peptide analogs for therapeutic and diagnostic applications, including radio-therapeutic and radio-diagnostic applications. In particular the present invention provides SST analogs backbone cyclized through metal complexation useful for scintigraphic imaging. The novel analogs according to the present invention having high affinity to SST receptor subtypes associated with several types of cancers, may be used for diagnosis and treatment of tumors by application of receptor-specific reagents.

Specific embodiments comprise somatostatin analog of three to twenty-four amino acids that incorporates at least one building unit, comprising N′-(o-functionalized derivative of an amino acid, wherein a backbone cyclic structure is formed by metal complexation to a chelating moiety comprising the at least one building unit and a second moiety selected from the group consisting of a second building unit, the side chain of an amino acid residue of the sequence or a terminal amino acid residue.

Distinct from native SST and SST analogs known in the art, the cyclic peptides of the present invention are SST analogs backbone cyclized through metal complexation, which possess unique and superior properties such as chemical and metabolic stability, selectivity, increased bioavailability and improved pharmacokinetics. These analogs are labeled with isotopes preferably radioisotopes used for cyclizing the peptide.

According to the present invention, novel labeled peptide analogs which are characterized in that they incorporate novel building units with bridging groups attached to the alpha nitrogens of alpha amino acids, are disclosed. Specifically, these compounds are backbone cyclized somatostatin analogs comprising a peptide sequence of three to twenty four amino acids, each analog incorporating at least one building unit, said building unit containing one nitrogen atom of the peptide backbone connected to a bridging group comprising a chelator-metal complex, preferably an N₂S₂ oxorhenium(V) or oxotechnetium(V) metal complex, wherein at least one building unit is connected via said bridging group to form a cyclic structure with a moiety selected from the group consisting of a second building unit, the side chain of an amino acid residue of the sequence or a terminal amino acid residue. Preferably, the peptide sequence incorporates 3 to 24 residues, more preferably 4 to 12 amino acids, most preferably 5-9 amino acids.

The present invention provides for the first time somatostatin analogs cyclized through site-specific metal complexation. The chelating of the metal to the peptide through binding to a chelating moiety coupled to at least one Na substituted amino acid, enables formation of a cyclic structure. In preferred embodiments the metal binds the peptide through a N₂S₂ type chelator. In most preferred embodiments, the chelator is built from two thiol groups of cysteine residues and two nitrogen atoms.

The diagnostic radiopharmaceutical comprising a peptide cyclized through a radionuclide has several distinct advantages over compounds known in the art that are already cyclic prior to metal complexation. In both cases the cold kit labeling process results in less than 10% of the kit peptide being complexed with metal. In the case of a cyclic non-metal/non-radioactive peptide, the peptide is relatively stable metabolically; this results in administration of a relatively long-circulating pharmacologically active compound. According to the present invention, the unlabelled linear peptide is expected to be unstable metabolically, therefore the 90% of unlabelled material should be cleared from the body rapidly and is expected to exhibit little to no pharmacological activity in comparison to analogs that are unlabeled cyclic species.

According to the present invention it is now disclosed that preferred labelled somatostatin analogs are analogs with improved affinity and selectivity to specific somatostatin subtypes. Preferred analogs include novel backbone cyclic analogs of somatostatin which display receptor selectivity to SST-R subtypes 2 or 5 or to SST-R subtypes 2 and 5. Other preferred analogs bind to more than two SST receptors.

Other preferred somatostatin analogs according to the present invention may advantageously incLude bicyclic structures containing at least one backbone structure cyclized through metal complexation, wherein at least one building unit is involved in the cyclic structure, and a second cyclic structure which is selected from the group consisting of side-chain to side-chain, backbone to backbone and backbone to terminal.

The invention further provides peptide reagents capable of being labelled to form backbone cyclic diagnostic and therapeutic agents. These reagents comprise a somatostatin analog covalently linked to a binding moiety which is formed using at least one N^{α}-ω-functionalized derivative of an amino acid. The metal binds to the binding moiety to form a backbone cyclic structure. In preferred embodiments according to the present invention the chelating moiety comprises four donor atoms and the metal is a radioactive isotope. According to a preferred embodiment of the present invention the chelator is built from two free thiols and two free nitrogens, which through complexation with a metal form a backbone cyclic structure. In most preferred analogs the chelator is made from two cysteine residues. In preferred embodiments according to the present invention at least one of the cysteine residues is covalently connected to the bridging group of an N^{α}-ω-functionalized derivative of an amino acid.

Preferred chelating moieties according to the present invention include those in which the four donor atoms are two nitrogens and two sulfurs (N₂S₂) and, through metal complexation, the peptide analog is cyclized and stable 5- to 6-membered rings are formed according to the general Formula No. 1: 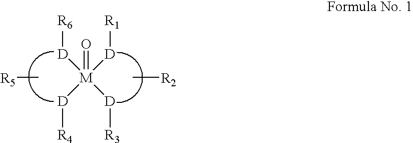
wherein the Ds represent the four donor atoms of N₂S₂;
the half-circles represent two- or three-carbon bridges between the donor atoms;
the R groups are independently selected from the group consisting of cyclic peptide, linear peptide, oxo, hydroxy, a hydrocarbon, hydrogen, a linking or spacing group connecting the peptide analog and the chelating moiety, and are located on a position selected from the donor atoms and the carbon bridges, wherein at least two of the R groups together with the chelating moiety form a cyclic peptide structure; and M is a metal atom preferably selected from Re and Tc in the +5 oxidation state.

Chelators of the N₂S₂ type are, for example, constructs of two NS hemi-chelators: two Cys residues; one Cys and one amidomercaptoacetyl (AMA) residue, one Cys and one amidomercaptoethyl (ANIE) residue; two AMA residues; one AMA and one AME residue; or two AME residues. The Cys residues is selected from the D and L stereoisomers and interposition of dissimilar residues on the peptide provides a second, isomeric analog.

In preferred analogs the peptide is coupled to one hemi-chelator via a linker and a second hemi-chelator via the peptide backbone, to form a structure of the general Formula No. 2:
Z-Q-PTR-X Formula No. 2
wherein Z is a first hemi-chelating moiety comprising two donor atoms, one N and one S, that through metal complexation form a five- to six-membered ring;
Q is absent or a linker moiety which can be coupled to a free functional group of the peptide; PTR denotes a somatostatin analog comprising at least one N-o)-functionalized derivative of an amino acid; and
X is a second hemi-chelating moiety comprising two donor atoms, one N and one S, that through metal complexation form a five- to six-membered ring, wherein the chelating moiety is linked through a lower alkyl chain comprising 1-6 carbon atoms, to the alpha nitrogen of the PTR backbone or to a free functional group of the peptide.

Preferably, the linker Q is connected to the N-terminal of the peptide, and X is connected to the peptide backbone or to a peptide side chain. More preferably the linker Q is absent or is selected from the group consisting of gamma amino butyric acid (GABA), Gly, and βAla, and X is connected to the α-nitrogen of an N-building unit. Most preferably, Z and X are each independently selected from the group consisting of L and D cysteines.

Some of the preferred analogs according to the present invention may comprise two or more isomers. The present invention includes such isomers either in combination or individually isolated.

The invention provides radiolabelled backbone cyclic peptides that are scintigraphic imaging agents, radiodiagnostic agents and radiotherapeutic agents.

Scintigraphic imaging agents of the invention comprise peptide reagents backbone cyclized through metal complexation with radionuclides, preferably ^{99m}Tc, for use in diagnostic imaging (single photon emission computed tomography, gamma camera, planar detector probes or devices for intraoperative use, positron emission tomography).

Radiotherapeutic agents of the invention comprise backbone cyclic peptide reagents radiolabelled with a cytotoxic radioisotope (having α or β emission). The most preferred cytotoxic radioisotopes according to the present invention are rhenium-186 and rhenium-188. Additional preferred radionuclides according to the invention are radioisotopes of indium, yttrium, lutetium, gallium and gadolinium. Combination embodiments, wherein a particular complex is useful both in scintigraphic imaging and in targeted radiotherapy, are also provided by the invention. Methods for making and using such backbone cyclic peptides, backbone cyclic reagents and radiolabelled embodiments thereof are also provided.

The currently most preferred SST analogs backbone cyclized through metal complexation according to the present invention are now disclosed: One embodiment is a compound having the general Formula No. 3 (SEQ ID NO: 1): 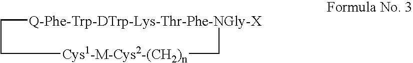
wherein n is 1 to 6;
Q is absent or is selected from the group consisting of GABA, Gly, and βAla;
X designates a terminal carboxy acid, amide or alcohol group;
Cys¹ and Cys² are each independently L or D isomers; and
M is a metal.
   Preferably:
n is 2, 3, or 6;
Q is absent or is βAla;
Cys² is LCys;
X is an amide; and
M is a radiometal selected from the group consisting of [^{nat}Re]oxorherium(V), [¹⁸⁶Re]oxorhenium(V), [¹⁸⁸Re]oxorhenium(V) or [^{99m}Tc]oxotechnetium(V).

Most preferred analogs according to formula 3 are selected from the group consisting of:
ReO-LCys*-Phe-Trp-DTrp-Lys-Thr-Phe-GlyN3(LCys*)-NH₂ denoted ReO-GF-29;
ReO-DCys*-Phe-Trp-DTrp-Lys-Thr-Phe-GlyN3(LCys*)-NH₂ denoted ReO-GF-31;
ReO-LCys*-βAla-Phe-Trp-DTrp-Lys-Thr-Phe-GlyN3(LCyS*)NH₂ denoted ReO-GF-21;
ReO-LCys*-βAla-Phe-Trp-DTrp-Lys-Thr-Phe-GlyN2(LCys*)-NH₂ denoted ReO-GF-37;
ReO-LCys*-Gly-Phe-Trp-DTrp-Lys-Thr-Phe-GlyiN6(DCys*)-NH₂ denoted ReO-GF-10;
ReO-DCys*-Gly-Phe-Trp-DTrp-Lys-Thr-Phe-GlyN6(LCys*)-NH₂ denoted ReO-GF-11;
ReO-LCys*-Ala-Phe-Trp-DTrp-Lys-Thr-Phe-GlyN6(DCys*)-NH₂ denoted ReO-GF-06;
ReO-DCys*-GABA-Phe-Trp-DTrp-Lys-Thr-Phe-GlyN6(LCys*)-NH₂ denoted ReO-GF-03;
ReO-DCys*-βAla-Phe-Trp-DTrp-Lys-Thr-Phe-GlyN6(DCys*)-NH₂ denoted ReO-GF-08;
ReO-LCys*-Phe-Trp-DTrp-Lys-Thr-Phe-GlyN6(DCys*)-NH₂ denoted ReO-GF-14;
ReO-LCys*-GABA-Phe-Trp-DTrp-Lys-Thr-Phe-GlyN6(DCys*)-NH₂ denoted ReO-GF-02;
ReO-DCys*-Gly-Phe-Trp-DTrp-Lys-Thr-Phe-GlyN6(DCys*)-NH₂ denoted ReO-GF-12;
   wherein the asterisks denote the chelating groups used for cyclization through metal complexation.

These backbone cyclized SST peptide analogs are prepared by incorporating at least one N^{α}-ω-functionalized derivative of an amino acid into a peptide sequence. Two hemi-chelating NS donor atom-containing moieties are added, one to the nitrogen of the N^{α}-ω-functionalized amino acid (for example through addition of Cys) and another to either the terminal N or to a straight-chain AA spacer at the N-terminus (for example through addition of Cys to the terminal N). Selective cyclization is accomplished through binding of a single metal or radiometal (preferably as oxorhenium(V) or oxotechnetium(V)) to both bidentate hemi-chelators to form a tetradentate N₂S₂ oxometal(V) cyclic peptide (or peptidomimetic) complex. The hemi-chelating moieties can alternatively be covalently bound to two N^{α}-ω-functionalizations, one or more amino acid side chain in the peptide sequence, or any combination of N^{α}-ω-functionalization, amino acid side chain, C- or N-terminus or linker or spacer group attached to any of the above.

It is another advantage of the SST analogs provided by this invention that the backbone cyclic linkage acts to protect the peptide from degradation byexopeptidases.

Somatostatin analogs backbone cyclized through metal complexation of the present invention may be used as diagnostic compositions in methods for diagnosing cancer and imaging the existence of tumors or their metastases, and in detection of allograft rejection including but not limited to cardiac allograft rejection. The methods for diagnosis of cancer and allograft rejection comprise administering to a mammal, including a human patient, a backbone cyclic analog or analogs labeled with a detectable tracer which is selected from the group consisting of a radioactive isotope and a non-radioactive tracer. The methods for the diagnosis or imaging of cancer and allograft rejection using such compositions represent another embodiment of the invention.

The pharmaceutical compositions comprising pharmacologically active labelled backbone cyclized SST agonists or antagonists and a pharmaceutically acceptable carrier or diluent represent another embodiment of the invention, as do the methods for the treatment of cancers in targeted radiotherapy using such compositions. The pharmaceutical compositions according to the present invention advantageously comprise at least one SST peptide analog backbone cyclized through metal complexation, which is selective for one or more SST receptor subtypes. These pharmaceutical compositions may be administered by any suitable route of administration, including orally, topically or systemically. Preferred modes of administration include but are not limited to parenteral routes such as intravenous and intramuscular injections, as well as via intra-nasal administration or oral ingestion.

The invention further provides a method for treating or diagnosing somatostatin-related diseases in animals, preferably humans, comprising administering a therapeutically effective amount of backbone cyclic SST analogs of the invention. In some preferred embodiments, the reagent is radioactively labeled with ¹⁸⁶Re or ¹⁸⁸Re.

Another aspect of the present invention provides methods for preparing therapeutic and diagnostic agents, including preferably scintigraphic imaging agents. Each such reagent comprises a SST analog capable of being backbone cyclized through metal complexation. The invention further provides kits for making and labelling such compositions.

### BRIEF DESCRIPTION OF THE DRAWINGS

FIG. 1 describes the synthetic scheme of the set of 48 somatostatin analogs backbone cyclized through metal complexation, synthesized.

FIG. 2 demonstrates the affinity of selected compounds, ReO-GF-21 and ReO-GF-31, to human SST-R2, measured by inhibition of the reference compound ¹²⁵I-Tyr¹¹-SRIF-14.

### DETAILED DESCRIPTION OF THE PREFERRED EMBODIMENTS

According to the present invention, peptide analogs are cyclized through metal complexation, via bridging groups attached to the alpha nitrogens of amino acids that permit novel non-peptidic linkages. In general, the procedures utilized to construct such peptide analogs from their building units rely on the known principles of peptide synthesis; most conveniently, the procedures can be performed according to the known principles of solid phase peptide synthesis.

The methods for design and synthesis of backbone cyclized analogs according to the present invention are disclosed in U.S. Pat. Nos. 5,811,392; 5,874,529; 5,883,293; 6,051,554; 6,117,974; 6,265,375, and international applications WO 95/33765; WO 97/09344; WO 98/04583; WO 99/31121; WO 99/65508; WO 00/02898; WO 00/65467 and WO 02/062819. All of these methods are incorporated herein in their entirety, by reference.

The most striking advantages of backbone cyclization are:
1) cyclization of the peptide sequence is achieved without compromising any of the side chains of the peptide thereby decreasing the chances of sacrificing functional groups essential for biological recognition (e.g. binding to specific receptors) and function.
2) optimization of the peptide conformation is achieved by allowing permutation of the bridge length, and bond type (e.g., amide, disulfide, thioether, thioester, urea, carbamate, or sulfonamide, etc.), bond direction, and bond position in the ring.
3) when applied to cyclization of linear peptides of known activity, the bridge can be designed in such a way as to minimize its interaction between the active region of the peptide and its cognate receptor. This decreases the chances of the cyclization arm interfering with recognition and function, and also creates a site suitable for attachment of tags such as radioactive tracers, cytotoxic drugs, photoactive substances, or any other desired label.

Distinct from native SST and SST analogs known in the background art, the peptides of the present invention are SST analogs backbone cyclized through metal complexation, which possess unique and superior properties such as chemical and metabolic stability, selectivity, increased bioavailability and improved pharmacokinetics. These analogs are labeled with metal isotopes, preferably radioisotopes.

The diagnostic radiopharmaceutical comprising a peptide cyclized through a radionuclide has several distinct advantages over compounds known in the art that are already cyclic prior to metal complexation. In both cases the cold kit labeling process results in less than 10% of the kit peptide being complexed with metal. In the case of a cyclic non-metal/non-radioactive peptide, the peptide is relatively stable metabolically; this results in administration of a relatively long-circulating pharmacologically active compound. According to the present invention, the unlabelled linear peptide is expected to be unstable metabolically, therefore the 90% of unlabelled material should be cleared from the body rapidly and is expected to exhibit little to no pharmacological activity in comparison to analogs that are unlabeled cyclic species.

Terminology and Definitions

The term “agonist of somatostatin” preferably means that the molecules are capable of mimicking at least one of the actions of somatostatin.

The term “antagonist of somatostatin” in the context of the present invention preferably means that these molecules are able to reduce or prevent at least one of the actions of somatostatin.

The term linker denotes a chemical moiety whose purpose is to link, covalently, a chelating moiety and a peptide, peptide analog or peptido-mimetic. The linker may be also used as a spacer whose purpose is to allow distance between the chelating moiety (thus the metal) and the peptide, peptide analog or peptido-mimetic.

The term “chelating agent” as used herein denotes a chemical moiety whose purpose is to stably form a chelating agent (or chelator)-metal complex. The complex is formed through electron donation from certain electron-rich atoms on the chelating agent to the electron-poor metal. The chelating agent typically has four donor atoms. The preferred donor atom for oxorhenium(V) and oxotechnetium(V) is nitrogen and the most preferred donor atom is sulfur.

“Hemi-chelator” denotes a chemical moiety whose purpose is to form half of the metal-complex with two donor atoms as described above. A second hemi-chelator on the same compound forms the second half of the complex with the same metal.

The term “scintigraphic imaging agent” as used herein is meant to encompass a radiolabelled agent capable of being detected with a radioactivity detecting means (including but not limited to a planar camera, a gamma-camera, a single photon emission (computed) tomography (SPECT or SPET) or any hand-held probe (e.g. Geiger-Muller counter or a scintillation detector) or device for use intraoperatively or otherwise in the detection of tumors.

As used herein “peptide” indicates a sequence of amino acids linked by peptide bonds. The peptides according to the present invention comprise a sequence of 3 to 24 amino acid residues, preferably 4 to 12 residues, more preferably 5 to 9 amino acids. A peptide analog according to the present invention may optionally comprise at least one bond which is an amide-replacement bond such as urea bond, carbamate bond, sulfonamide bond, hydrazine bond, or arty other covalent bond.

The term “analog” further indicates a molecule which has the amino acid sequence according to the invention except for one or more amino acid changes. The design of appropriate “analogs” may be computer assisted.

Whenever “peptide of the invention” or “analogs of the invention” are mentioned in the present specification and claims, also salts and functional derivatives thereof are contemplated, as long as the biological activity of the peptide with respect to SST is maintained. Salts of the peptides of the invention contemplated by the invention are physiologically acceptable organic and inorganic salts. Functional derivatives of the peptides of the invention covers derivatives which may be prepared from the functional groups which occur as side chains on the residues or the N- or C-terminal groups, by means known in the art, and are included in the invention as long as they remain pharmaceutically acceptable, i.e., they do not destroy the activity of the peptide and do not confer toxic properties on compositions containing it. These derivatives may, for example, include aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or carbocyclic aroyl groups) or O-acyl derivatives of free hydroxyl group (for example that of seryl or threonyl residues) formed by reaction with acyl moieties.

As used herein the term “backbone cyclic peptide” or “backbone cyclic analog” denotes an analog of a linear peptide comprising a peptide sequence of preferably 3 to 24 amino acids that incorporates at least one building unit, comprising N-co-functionalized derivative of an amino acid, wherein
i. said building unit containing one nitrogen atom of the peptide backbone connected to a bridging group comprising an amide, thioether, thioester, disulfide, urea, carbamate, or sulfonamide, wherein at least one building unit is connected via said bridging group to form a cyclic structure with a moiety selected from the group consisting of a second building unit, the side chain of an amino acid residue of the sequence or a terminal amino acid residue; or
ii. a backbone cyclic structure is formed by metal complexation to a chelating moiety connected to at least one building unit and to a second moiety selected from the group consisting of a second building unit, the side chain of an amino acid residue of the sequence or a terminal amino acid residue.

More preferably, the peptide sequence incorporates 3-24 amino acids, still more preferably it incorporates 4-12 amino acids, and most preferably 5-9 amino acids.

A “building unit” indicates an N^{α} derivatized amino acid of general Formula No. 4: 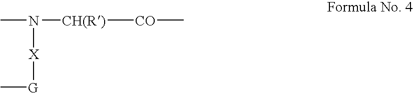
wherein X is a spacer group selected from the group consisting of alkylene, substituted alkylene, arylene, cycloalkylene and substituted cycloalkylene; R′ is an amino acid side chain, optionally bound with a specific protecting group; and G is a functional group selected from the group consisting of amines, thiols, alcohols, carboxylic acids, sulfonates and esters, and alkyl halides; which is incorporated into the peptide sequence and subsequently selectively cyclized via the functional group G with one of the side chains of the amino acids in said peptide sequence or with another ω-functionalized amino acid derivative, via complexation with a metal or metal, through N₂S₂ donor chemistry.

The methodology for producing the building units is described in international patent applications published as WO 95/33765 and WO 98/04583 and in U.S. Pat. Nos. 5,770,687 and 5,883,293 all of which are expressly incorporated herein by reference thereto as if set forth herein in their entirety.

The building units are abbreviated by the three letter code of the corresponding modified amino acid followed by the type of reactive group (N for amine, C for carboxyl), and an indication of the number of spacing methylene groups. For example, GlyC2 describes a modified Gly residue with a carboxyl reactive group and a two carbon methylene spacer, and PheN3 designates a modified phenylalanine group with an amino reactive group and a three carbon methylene spacer. In generic formulae the building units are abbreviated as R with a superscript corresponding to the position in the sequence preceded by the letter N, as an indication that the backbone nitrogen at that position is the attachment point of the bridging group specified in said formulae.

The compounds herein disclosed may have asymmetric centers. All chiral, diasteromeric, and racemic forms are included in the present invention. Many geometric isomers of double bonds and the like can also be present in the compounds disclosed herein, and all such stable isomers are contemplated in the present invention. By “stable compound” or “stable structure” is meant herein a compound that is sufficiently robust to survive isolation to a useful degree of purity from a reaction mixture, and formulation into an efficacious diagnostic or therapeutic agent.

The term, “substituted” as used herein and in the claims, means that any one or more hydrogen atoms on the designated atom is replaced with a selection from the indicated group, provided that the designated atom's normal valency is not exceeded, and that the substitution results in a stable compound.

When any variable (for example R, X, Z, etc.) occurs more than one time in any constituent or in any Formula herein, its definition on each occurrence is independent of its definition at every other occurrence. Also, combinations of substituents and/or variables are permissible only if such combinations result in stable compounds.

As used herein and in the claims, the phrase “therapeutically effective amount” means that amount of novel backbone cyclized peptide analog or composition comprising same to administer to a host to achieve the desired results for the indications disclosed herein, such as but not limited to cancer, endocrine disorders, inflammatory diseases, and gastrointestinal disorders.

Certain abbreviations are used herein to describe this invention and the manner of making and using it. For instance, Alloc refers to allyloxycarbonyl, Boc refers to the t-butyloxycarbonyl, DCM refers to dichloromethane, DIEA refers to diisopropyl-ethyl amine, DMF refers to dimethyl formamide, DTPA refers to diethylenetriaminepentaacetic acid, Fmoc refers to fluorenylmethoxycarbonyl, HPLC refers to high pressure liquid chromatography, GABA refers to gamma aminobutyric acid, mCi refers to millicurie, MS refers to mass spectrometry, NMP refers to 1-methyl-2-pyrolidonone, PET refers to positron emission tomography, PyBrOP refers to bromo-tris-pyrrolidino-phosphonium hexafluorophosphate, SPECT refers to single photon emission computed tomography, SPET refers to single photon emission tomography, SRIF refers to Somatotropin Release Inhibitory Factor, SST refers to somatostatin, SST-R refers to somatostatin receptor, TFA refers to trifluoroacetic acid.

The amino acids used in this invention are those which are available commercially or are available by routine synthetic methods. Certain residues may require special methods for incorporation into the peptide, and sequential, divergent and convergent synthetic approaches to the peptide sequence are useful in this invention. Natural coded amino acids and their derivatives are represented by three-letter codes according to IUPAC conventions. When there is no indication, the L isomer was used. The D isomers are indicated by “(D)” or “D” before the residue abbreviation. List of Non-coded amino acids: Abu refers to 2-aminobutyric acid, Dab refers to diaminobutyric acid, Dpr and Dap both refer to diaminopropionic acid, GABA refers to gamma aminobutyric acid, 1 Nal refers to 1-naphthylalanine, 2Nal refers to 2-naphtylalanine, and Nle refers to norleucine.

Conservative substitution of amino acids as known to those skilled in the art is within the scope of the present invention. Conservative amino acid substitutions includes replacement of one amino acid with another having the same type of functional group or side chain e.g. aliphatic, aromatic, positively charged, negatively charged. These substitutions may enhance oral bioavailability, penetration into the central nervous system, targeting to specific cell populations and the like. One of skill will recognize that individual substitutions, deletions or additions to peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).
   Pharmacology

Apart from other considerations, the fact that the novel active ingredients of the invention are peptides or peptide analogs, dictates that the formulation be suitable for delivery of these type of compounds. Clearly, peptides are less suitable for oral administration due to susceptibility to digestion by gastric acids or intestinal enzymes. The preferred routes of administration of peptides are intra-articular, intravenous, intramuscular, subcutaneous, intradermal, or intrathecal. A more preferred route is by direct injection at or near the site of disorder or disease.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

For injection, the compounds of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants for example polyethylene glycol are generally known in the art.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration. For buccal administration, the compositions may take the form of tablets or lozenges formulated in conventional manner.

For administration by inhalation, the variants for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the peptide and a suitable powder base such as lactose or starch.

Pharmaceutical compositions for parenteral administration include aqueous solutions of the active ingredients in water-soluble form. Additionally, suspensions of the active compounds may be prepared as appropriate oily injection suspensions. Suitable natural or synthetic carriers are well known in the art (Pillai et al., Curr. Opin. Chem. Biol. 5:447, 2001). Optionally, the suspension may also contain suitable stabilizers or agents, which increase the solubility of the compounds, to allow for the preparation of highly concentrated solutions. Alternatively, the active ingredient may be in powder form for reconstitution with a suitable vehicle, e.g., sterile, pyrogen-free water, before use.

The compounds of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of a compound effective to prevent, alley iate or ameliorate symptoms of a disease of the subject being treated. Determination of a therapeutically effective amount is well within the capability of those skilled in the art.

Toxicity and therapeutic efficacy of the peptides described herein can be determined by standard pharmaceutical procedures in cell cultures or experimental animals, e.g., by determining the IC₅₀ (the concentration which provides 50% inhibition) and the LD₅₀ (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be (c osen by the individual physician in view of the patient's condition (e.g. Fungi, et al., 1975, in “The Pharmacological Basis of Therapeutics”, Ch. 1 p. 1).

Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, and all other relevant factors.

### Preferred Embodiments

According to the present invention, novel labelled peptide analogs which are characterized in that they incorporate novel building units with bridging groups attached to the alpha nitrogens of alpha amino acids, are disclosed. Specifically, these compounds are backbone cyclized somatostatin analogs comprising a peptide sequence of three to twenty four amino acids, each analog incorporating at least one building unit, said building unit containing one nitrogen atom of the peptide backbone connected to a bridging group comprising an N₂S₂ oxorhenium(V) or oxotechnetium(V) metal complex, wherein at least one building unit is connected via said bridging group to form a cyclic structure with a moiety selected from the group consisting of a second building unit, the side chain of an amino acid residue of the sequence or a terminal amino acid residue. Preferably, the peptide sequence incorporates 3 to 24 residues, more preferably 4 to 12 amino acids, most preferably 5-9 amino acids.

Backbone cyclic analogs of the present invention bind with high affinity to a defined subset of the human SST receptors. This receptor selectivity indicates the potential physiological selectivity in vivo. Furthermore, the present invention provides for the first time the possibility to obtain a panel of backbone cyclized labelled analogs with specific SST receptor selectivity or with combinations of receptor selectivity. This enables diagnostic and therapeutic uses in different types of cancers according to the specific needs of each patient and each disease.

According to the present invention it is now disclosed that preferred SST analogs are nonapeptide analogs backbone cyclized through metal complexation, with improved affinity and selectivity to specific SST subtypes. Preferred analogs include novel backbone cyclic analogs of SST which may display receptor selectivity to SST-R subtypes 2 or to SST-R subtypes 2 and 5.

Other preferred somatostatin analogs according to the present invention may advantageously include bicyclic structures containing at least one backbone structure cyclized through metal complexation, wherein at least one building unit is involved in the cyclic structure, and a second cyclic structure which is selected from the group consisting of side-chain to side-chain; backbone to backbone and backbone to terminal.

The invention further provides peptide reagents capable of being labelled to form backbone cyclic diagnostic and therapeutic agents. These reagents comprise a somatostatin analog covalently linked to a metal-binding moiety which is formed using at least one N-co-functionalized derivative of an amino acid. The metal binds to the metal-binding moiety to form a backbone cyclic structure. In preferred embodiments according to the present invention the chelating moiety comprises four donor atoms and the metal is comprises radioactive isotope. According to the present invention the chelator is built from two free thiols and two free nitrogens, which through complexation with a metal form a backbone cyclic structure. In most preferred analogs the chelator is made from two cysteine residues. In preferred embodiments according to the present invention at least one of the Cysteine residues is covalently connected to the bridging group of an N^{α}-ω-functionalized derivative of an amino acid.

Preferred chelating moieties according to the present invention include those in which the four donor atoms are two nitrogens and two sulfurs (N₂S₂) and, through metal complexation, the peptide analog is cyclized and stable 5- to 6-membered rings are formed according to the general Formula No. 1: 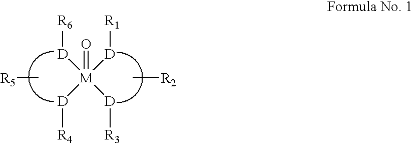
wherein the Ds represent the four donor atoms of N₂S₂;
the half-circles represent two- or three-carbon bridges between the donor atoms;
the R groups are independently selected from the group consisting of cyclic peptide, linear peptide, oxo, hydroxy, a hydrocarbon, hydrogen, a linking or spacing group connecting the peptide analog and the chelating moiety, and are located on a position selected from the donor atoms and the carbon bridges, wherein at least two of the R groups together with the chelating moiety form a cyclic peptide structure; and M is a metal atom preferably selected from Re and Tc in the +5 oxidation state.

Additional preferred embodiments comprise chelating moieties to form oxorhenium(V) or oxotechnetium(V) complexes having −1, neutral, +1, or +2 electronic charges as described in the following table:

TABLE NO. 1
N₂S₂ donor set description

| | Oxo-metal(V) charge |
|---|---|
| Donor chemical descriptor | when complexed |
| Amide-amide-sulfhydryl-sulfhydryl | −1 |
| Amide-amine-sulfhydryl-sulfhydryl | Neutral |
| Amine-amine-sulfhydryl-sulfhydryl | +1 |

The invention provides radiolabelled backbone cyclic peptides that are scintigraphic imaging agents, radiodiagnostic agents and radiotherapeutic agents.

Scintigraphic imaging agents of the invention comprise backbone cyclic peptide reagents radiolabelled with gamma-radiation emitting isotopes, preferably ^{99m}Tc for use in diagnostic imaging (single photon emission computed tomography, gamma camera, planar, detector probes or devices for intraoperative use). Any other technetium or rhenium radioisotopes having decay characteristics making them useful in radionuclide imaging (including positron emission tomography, PET), capable of complexation with the backbone cyclic analogs of the invention, are also encompassed by the present invention.

Radiotherapeutic agents of the invention comprise backbone cyclic peptide reagents radiolabelled with a cytotoxic radioisotope (a or P emission). Most preferred cytotoxic radioisotopes according to the present invention are rhenium-186 and rhenium-188. Combination embodiments, wherein such a complex is useful both in scintigraphic imaging and in targeted radiotherapy, are also provided by the invention. Any other technetium or rhenium radioisotopes having decay characteristics making them useful in radiotherapy, capable of complexation with the backbone cyclic analogs of the invention, are also are also encompassed by the present invention.

Somatostatin analogs backbone cyclized through metal complexation according to the invention may be also used as contrast agents for magnetic resonance imaging (MRI) of cancer. In proton MRI diagnostics, increased contrast of internal organs and tissues may be obtained by administrating compositions containing paramagnetic metal species, which increase the relaxivity of surrounding water protons. In addition, the compounds of the present invention may be used for computed tomography (CT) diagnostics wherein increased contrast of tumors is obtained by administering a contrast agent which is substantially radiopaque.

Somatostatin is a tetradecapeptide hormone whose numerous regulatory functions are mediated by a family of five receptors, whose expression is tissue dependent. Receptor specific analogs of SST are believed to be valuable diagnostic and therapeutic agents in the treatment and diagnosis of various diseases. Attempts to design small peptide analogs having this selectivity have not been fully successful. It has now unexpectedly been found that the SST analogs backbone cyclized through metal complexation, of the present invention, are highly selective to SST receptor subtypes and are therefore useful for diagnosis and treatment of conditions where specific SST receptors are expressed in specific tissues. Such conditions are preferably different types of cancers such as colon cancer, growth hormone-secreting pituitary adenoma, thyroid cancer, gastric carcinoid, small cell lung carcinoma, melanoma, medullary non-Hodgkin's lymphoma, and breast cancer and other types of cancer. In addition, the backbone cyclized SST analogs of the present invention may be used for detection of allograft rejection including but not limited to cardiac allograft rejection.

Backbone cyclized analogs of the present invention may be used as diagnostic compositions in methods for diagnosing cancer and imaging the existence of tumors or their metastases, and in detection of allograft rejection including but not limited to cardiac allograft rejection. The methods for diagnosis of cancer and allograft rejection comprise administering to a mammal, including a human patient, a backbone cyclic analog or analogs labeled with a detectable tracer which is selected from the group consisting of a radioactive isotope and a non-radioactive tracer. The methods for the diagnosis or imaging of cancer and allograft rejection using such compositions represent another embodiment of the invention.

The imaging agents provided by the invention have utility for tumor imaging, particularly for imaging primary and metastatic neoplastic sites wherein said neoplastic cells express SST receptors, and in particular such primary and especially metastatic tumor cells that have been clinically difficult to detect and characterize using conventional methodologies. The imaging reagents according to the present invention may be used for visualizing organs, and tumors, in particular gastrointestinal tumors, myelomas, small cell lung carcinoma and other APUDomas, endocrine tumors such as medullary thyroid carcinoma and pituitary tumors, brain tumors such as meningiomas and astrocytomas, and tumors of the prostate, breast, colon, and ovaries can also be imaged.

The ^{99m}Tc labeled diagnostic reagents are preferably administered intravenously in a single unit injectable dose. These reagents may be administered in any conventional medium for intravenous injection such as an aqueous saline medium. Generally, the unit dose to be administered has radioactivity of about 1 to 30 mCi. The solution to be injected at unit dosage is from about 0.1 to about 10 mL. After intravenous administration, imaging in vivo can be performed any time from immediately up to and including four physical decay half lives following administration. Any method of scintigraphic imaging such as gamma scintigraphy, can be utilized in accordance with the present invention.

Radioactively-labeled scintigraphic imaging agents according to the present invention are provided having radioactivity in solution containing at concentrations of from about 1 mCi to 100 mCi per mL.

The pharmaceutical compositions comprising pharmacologically active backbone cyclized SST agonists or antagonists and a pharmaceutically acceptable carrier or diluent represent another embodiment of the invention, as do the methods for the treatment of cancers in targeted therapy using such compositions. The pharmaceutical compositions according to the present invention advantageously comprise at least one backbone cyclized peptide analog which is selective for one or two SST receptor subtypes. These pharmaceutical compositions may be administered by any suitable route of administration, including orally, topically or systemically. Preferred modes of administration include but are not limited to parenteral routes such as intravenous and intramuscular injections, as well as via intra-nasal administration or oral ingestion. The preferred doses for administration of such pharmaceutical compositions range from about 0.1 μg/kg to about 20 mg/kg body weight/day.

The pharmaceutical compositions may preferably be used to promote regression of certain types of tumors, particularly those that express SST receptors. Furthermore, the pharmaceutical compositions can also be used to reduce the hormonal hypersecretion that often accompanies certain cancers, such as the APUDomas. Other conditions of which the compounds of the present invention are useful for treatment are endocrine disorders, gastrointestinal disorders, diabetes-associated complications, pancreatitis, autoimmune diseases, and inflammatory diseases, allograft rejection, atherosclerosis and restenosis.

The invention further provides a method for alleviating so matostatin-related diseases in animals, preferably humans, comprising administering a therapeutically effective amount of backbone cyclic SST analogs of the invention to the animal. In some preferred embodiments the backbone cyclic analog is unlabeled.

In some preferred embodiments, rhenium-186 or rhenium-188 may be used for radiotherapy of certain tumors if the reagent is radioactively labeled with cytotoxic radioisotopes such as ¹⁸⁶Re or ¹⁸⁸Re. In preferred embodiments, the amount of the SST analog administered is from about 0.1 μg/kg to about 20 mgikg body weight/day. For this purpose, an amount of radioactive isotope from about 10 mCi to about 200 mCi may be administered via any suitable clinical route, preferably by intravenous injection.

Another aspect of the present invention provides methods for preparing therapeutic and diagnostic pharmaceuticals, preferably scintigraphic imaging agents, and the reagents required to make them. Each such reagent is comprised of a SST analog covalently linked to a radiometal complexing moiety. For example, scintigraphic imaging agents provided by the invention comprise ^{99m}Tc labeled complexes formed by reacting the reagents of the invention with ^{99m}Tc in the presence of an agent capable of reducing [^{99m}Tc]pertechnetate ion (+7 metal oxidation state, that elutes from the ⁹⁹Mo/^{99m}Tc generator found commonly in the nuclear medicine clinic or nuclear pharmacy) to the oxo[^{99m}Tc]technetium species (+5 metal oxidation state). Preferred reducing agents include but are not limited to dithionite, stannous and ferrous ions. Such ^{99m}Tc complexes of the invention are also formed by labeling the peptide analogs of the invention with ^{99m}Tc by ligand exchange of a prereduced ^{99m}Tc complex. In this case, a weak chelator is present in the in situ reduction cocktail, but the reagents of this invention are not initially present. The reagents of this invention are then added to the solution containing the +5 oxidation state oxo[^{99m}Tc]technetium “weak chelator” complex, forming the more stable oxo[^{99m}Tc]technetium complex with the reagents of this invention.

The invention further provides kits for labelling SST analogs backbone cyclized through metal complexation. In a preferred embodiment of the invention, a kit for preparing [^{99m}Tc]technetium-labeled peptide analogs is provided. An appropriate amount of the backbone cyclic analog is introduced into a vial containing a reducing agent, such as stannous chloride, in an amount sufficient to label the analog with ^{99m}Tc. An appropriate amount of a transfer ligand (a weak oxo[^{99m}Tc]technetium chelator such as tartrate, citrate, gluconate, 2,5-dihydroxybenzoate, glucoheptanoate or mannitol, for example) can also be included. The kit may also contain additives such as salts to adjust the osmotic pressure, buffers to adjust the pH or preservatives to allow longer storage of either the cold kid or the final diagnostic radiopharmaceutical. The components of the kit may be in liquid, frozen or in dry form. In a preferred embodiment, the kit components are provided in lyophilized form.

Technetium-99m labeled imaging reagents according to the present invention may be prepared by the addition of an appropriate amount of ^{99m}Tc or ^{99m}Tc-complex into the vial containing the reagents according to the present invention, and reaction under appropriate conditions. Kits for preparing radiotherapeutic agents wherein the preferred radioisotopes are rhenium-186 and rhenium-188 are also provided.

### Most Preferred Embodiments

The most preferred backbone cyclized SST analogs according to the present invention are now described.

In preferred analogs the peptide is coupled to one hemi-chelator via a linker and a second hemi-chelator via the peptide backbone, to form a structure of the general Formula No. 2:
Z-Q-PTR-X Formula No. 2
wherein Z is a first hemi-chelating moiety comprising two donor atoms, one N and one S, that through metal complexation form a five- to six-membered ring;
Q is absent or a linker moiety which can be coupled to a free functional group of the peptide; PTR denotes a somatostatin analog comprising at least one N′-o)-functionalized derivative of an amino acid; and
X is a second hemi-chelating moiety comprising two donor atoms, one N and one S, that through metal complexation form a five- to six-membered ring, wherein the chelating moiety is linked through a lower alkyl chain comprising 1-6 carbon atoms, to the alpha nitrogen of the PTR backbone or to a free functional group of the peptide.

Preferably, the linker Q is connected to the N-terminal of the peptide, and X is connected to the peptide backbone or to a peptide side chain. More preferably the linker Q is absent or is selected from the group consisting of gamma amino butyric acid (GABA), Gly, and βAla, and X is connected to the α-nitrogen of an N-building unit. Most preferably, Z and X are selected from the group consisting of L and D cysteines. One embodiment is a compound having the general Formula No. 3 (SEQ ID NO: 1): 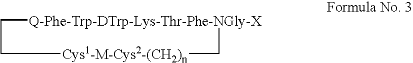
wherein n is 1 to 6;
Q is absent or is selected from the group consisting of GABA, Gly, and βAla;
X designates a terminal carboxy acid, amide or alcohol group;
Cys¹ and Cys² are each independently L or D isomers; and
M is a metal.
   Preferably:
n is 2, 3, or 6;
Q is absent or is βAla;
Cys² is LCys;
X is an amide; and
M is a radiometal selected from the group consisting of [‘a’ Re]oxorhenium(V), [¹⁸⁶Re]oxorhenium(V), [¹⁸⁸Re]oxorhenium(V) or [^{99m}Tc]oxotechnetium(V).

Most preferred analogs according to formula 3 are selected from the group consisting of:
ReO-LCys*-Phe-Trp-DTrp-Lys-Thr-Phe-GlyN3(LCys*)-NH₂ denoted ReO-GF-29;
ReO-DCys*-Phe-Trp-DTrp-Lys-Thr-Phe-GlyN3(LCys*)-NH₂ denoted ReO-GF-31;
ReO-LCys*-Ala-Phe-Trp-DTrp-Lys-Thr-Phe-GlyN3(LCys*)-NH₂ denoted ReO-GF-21;
ReO-LCys*-βAla-Phe-Trp-DTrp-Lys-Thr-Phe-GlyN2(LCys*)-NH₂ denoted ReO-GF-37;
ReO-LCys*-Gly-Phe-Trp-DTrp-Lys-Thr-Phe-GlyN6(DCys*)-NH₂ denoted ReO-GF-10;
ReO-DCys*-Gly-Phe-Trp-DTrp-Lys-Thr-Phe-GlyN6(LCys*)-NH₂ denoted ReO-GF-11;
ReO-LCys*-βAla-Phe-Trp-DTrp-Lys-Thr-Phe-GlyN6(DCys*)-NH₂ denoted ReO-GF-06;
ReO-DCys*-GABA-Phe-Trp-DTrp-Lys-Thr-Phe-GlyN6(LCys*)—NH₂ denoted ReO-GF-03;
ReO-yN6(DCys*)-NH₂ denoted ReO-GF-08;
ReO-LCys*-Phe-Trp-DTrp-Lys-Thr-Phe-GlyN6(DCys*)-NH₂ denoted ReO-GF-14;
ReO-LCys*-GABA-Phe-Trp-DTrp-Lys-Thr-Phe-GlyN6(DCys*)-NH₂ denoted ReO-GF-02;
ReO-DCys*-Gly-Phe-Trp-DTrp-Lys-Thr-Phe-GlyN6(DCys*)-NH₂ denoted ReO-GF-12;
   wherein the asterisks denote the chelating groups used for cyclization through metal complexation.

These backbone cyclized SST peptide analogs are prepared by incorporating at least one N^{α}-ω-functionalized derivative of an amino acid into a peptide sequence. Two hemi-chelating NS donor atom-containing moieties are added, one to the nitrogen of the N^{α}-ω-functionalized amino acid (for example through addition of Cys) and another to either the terminal N or to a straight-chain AA spacer at the N-terminus (for example through addition of Cys to the terminal N). Selective cyclization is accomplished through binding of a single metal or metal (preferably as oxorhenium(V) or oxotechnetium(V)) to both bidentate hemi-chelators to form a tetradentate N₂S₂ oxometal(V) cyclic peptide (or peptidomimetic) complex. The hemi-chelating moieties can alternatively be covalently bound to two N^{α}-ω-functionalizations, one or more amino acid side chain in the peptide sequence, or any combination of N^{α}-ω-functionalization, amino acid side chain, C- or N-terminus or linker or spacer group attached to any of the above.

Labelled derivatives of PTR 3173, according to the present invention are expected, like their parent, to bind both SST-R2 and SST-R5 and therefore may be used to detect and treat malignancies expressing both receptor types.

The affinity of the preferred analogs according to the present invention to type 2 SST receptor is in the subnanomolar-nanomolar range which makes these analogs potentially effective diagnostic and therapeutic compositions.

General Method for Synthesis, Purification and Characterization of SST Analogs Backbone Cyclized through Metal Complexation

Analogs were synthesized using the “Tea-bag” method modified form IHoughten R., (Proc. Natl. Acad. Sci. U.S.A. 82, 5131-5135, 1985), as hereinbelow:
Resin: 9.6 g of Rink amide MBHA resin, loading of 0.55 mmol/g was placed in 48 polypropylene bags (“Tea bags”) 4 cm×5 cm in size, such to have 0.2g of resin in each bag. The bags were placed in four plastic containers, 12 bags in each one.
Fmoc-deprotection: With 50 mL of 20% piperidine in NMP (twice for 30 minutes), followed by 5 washes with 50 mL NMP and 3 washes with 50 mL DCM each for two minutes with shaking.
   Couplings:
i. Regular couplings: with a solution containing 3 equivalents aino acid, 3 equivalents PyBroP and 7 equivalents of DIEA in 50 mL NMP. For 2 hours with shaking. Coupling is monitored by ninhydrin test and repeated until the ninhydrin solution remains yellow.
ii. Coupling to Gly building unit: with a solution containing 5 equivalents amino acid, 5 equivalents PyBroP and 12 equivalents of DIEA in 50 mL NMP. Twice for 2 hours with shaking.
Removal of the Alloc protectina crroup of the building units: with 0.6 equivalents per Alloc of Pd(PPh₃)₄ in 30 mL DCM containing 5% acetic acid and 2.5% methylmorpholine. For 1-4 hours with shaking in the dark.
Coupling of Boc-(L or D)Cys(Trt)-OH: following regular coupling protocol described above.
Fmoc-deprotection: following regular Deprotection protocol described above.
Coupling of Boc-(L or D)Cys(Trt)-OH: following regular coupling protocol described above.
Cleavage: with 95% TFA supplemented with scavengers: 2.5% H₂O and 2.5% triisopropylsilane.
Cyclization: performed after the peptides are cleaved from the resin and dissolved in 2 mL water with a solution containing 1 equivalent of Trichlorooxobis(triphenyl-phosphine) rhenium (v) in DMF (1/1 mL per mg peptide, total volume). For 1-3 hours with shaking. Cyclization is monitored by analytical HPLC.
Purification: An individual purification method for each backbone cyclic peptide is developed on analytical HPLC to optimize isolation of the cyclic peptide from other components. The analytical method is usually performed using a C-18 Vydac column 250×4.6 mm as the stationary phase and water/acetonitrile containing 0.1% TFA mixture gradient. The preparative method is designed by adapting the analytical separation method to the preparative C-18 Vydac column. The collected fractions are injected to the analytical HPLC to check purity. The pure fractions are combined and lyophilized.
Characterization: The combined pure lyophilized material is analyzed for purity by HPLC, MS and capillary electrophoresis and by amino acid analysis for peptide content and amino acid ratio determination.
   General Methods for Radiolabelling with Technetium

In forming a complex of radioactive technetium with the reagents of this invention, the ⁹⁹Mo/^{99m}Tc generator eluent, preferably containing sodium [^{99m}Tc]pertechnetate (+7 oxidation state), is reacted with the reagent in the presence of a reducing agent. The preferred reducing agent is stannous chloride, which reliably reduces Tc^{VII} to Tc^{V}. Means for preparing such complexes are conveniently provided in a kit form comprising a sealed vial containing a predetermined quantity of a reagent of the invention to be labeled and a sufficient amount of reducing agent to label the reagent with Tc-99m. Alternatively, the complex may be formed by reacting a reagent of this invention with a pre-formed labile complex of technetium and another compound known as a transfer ligand. This process is known as ligand exchange and is well known to those skilled in the art. The labile complex may be formed using such transfer ligands as tartrate, citrate, gluconate, 2,5-dihydroxybenzoate, glucoheptanoate or mannitol, for example.

General Method for Forming Metal Complexes with Crude Chelator-Cyclic Peptide Conjugates:

Crude chelator peptide conjugates can be complexed with oxorhenium(V). The post-cleavage crude is weighed and the molar amount is calculated, assuming the mass is 100% desired conjugate. Alternatively, the molar amount of conjugate is calculated based on the solid phase resin loading. The appropriate metal reagent is added at an equimolar amount. This strategy works with rhenium when the crude peptide is relatively pure. By avoiding a chromatographic purification step, time and resources are saved.

General Method for In Vitro Screening of Somatostatin Analogs

The ability of the SST analogs of the invention to bind to SST receptors in vitro was demonstrated by assaying the ability of such analogs to inhibit binding of a radiolabelled SST analog to SST receptor-containing cell membranes.

The SST analogs were tested for their potency in inhibition of the binding of ¹²⁵I-Tyr¹¹-SRIF (based on the method described by Raynor et. al., Molecular Pharmnacology 43: 838, 1993) to membrane preparations expressing the transmembranal SST receptors (SST-R1, 2, 3, 4 or 5). The receptor preparations used for these tests were either from the cloned human receptors selectively and stably expressed in Chinese Hamster Ovary (CHO) cells or from cell lines naturally expressing the SST-R5. Typically, cell membranes were homogenized in Tris buffer in the presence of protease inhibitors and incubated for 30-40 minutes with ¹²⁵I-Tyr¹¹-SRIF with different concentrations of the tested sample. The binding reactions were filtered, the filters were washed and the bound radioactivity was counted in β counter after addition of scintillation colution. Non specific binding was defined as the radioactivity remaining bound in the presence of 1 μM unlabeled SRIF-14.

In Vivo Models for Evaluating the Activity of Somatostatin Analogs

The radiolabeled compounds of the present invention are tested in vivo for tumor uptake in xenografts derived from cell lines such as the following:
i. Rat pituitary adcnoma cells (GH3) in nude rats.
ii. Human colon adenocarcinoma cells (HT-29) in nude mice or nude rats.
iii. Rat pancreatic acinar carcinoma cells (CA20948) in normal rats.
iv. Rat pancreatic cancer cells (AR42J) in nude mice. V. Human small cell lung carcinoma cells (NCI-H69) in nude mice.
vi. Human pancreatic carcinoid cells (BON-1) in nude mice or nude rats.
vii. LCC-18 cells in nude mice or nude rats.

Briefly, the cells are implanted intramuscularly in a suspension of 0.05 to 0.1 mL/animal, the tumors are allowed to grow to approximately 0.5 to 2 g, harvested, and used to implant a second, naive set of animals. Passaging in this fashion is repeated to generate successive generations of tumor-bearing animals. Third- to fifth-passage of tumor-bearing animals are injected intravenously with labeled compound. At selected times, the animals are sacrificed and harvested tissue samples are weighed and counted, along with an aliquot of the injected dose, in a gamma well-counter. Alternatively, the radiolabelled compounds are studied in normal or immuno-deficient-tumor-free animals. For example, in such in-vivo study, SST-R target uptake is monitored in the pancreas and adrenal, and the non-target organs are also monitored to ascertain each compound's clearance profile.

General In Vivo Imaging Methods

In vivo imaging of SST receptors expressed by animal tumor cells is performed essentially as described by Bakker et al. (1991, Life Sciences 49:1593-1601). Additional in vivo screening methods are described in details in Examples 6.

Conformationally constrained SST analogs constructed based in part on the sequences of a number of known biologically active peptides or based on previously unknown novel sequences are presented in the examples below. The following examples are intended to illustrate how to make and use the compounds and methods of this invention and are in no way to be construed as a limitation. Although the invention will now be described in conjunction with specific embodiments thereof, it is evident that many modifications and variations will be apparent to those skilled in the art. Accordingly, it is intended to embrace all such modifications and variations that fall within the spirit and broad scope of the amended claims.

The invention will now be illustrated in a non-limitative manner by the following

### EXAMPLES

### Example 1

### Detailed Procedure of Synthesis of Library

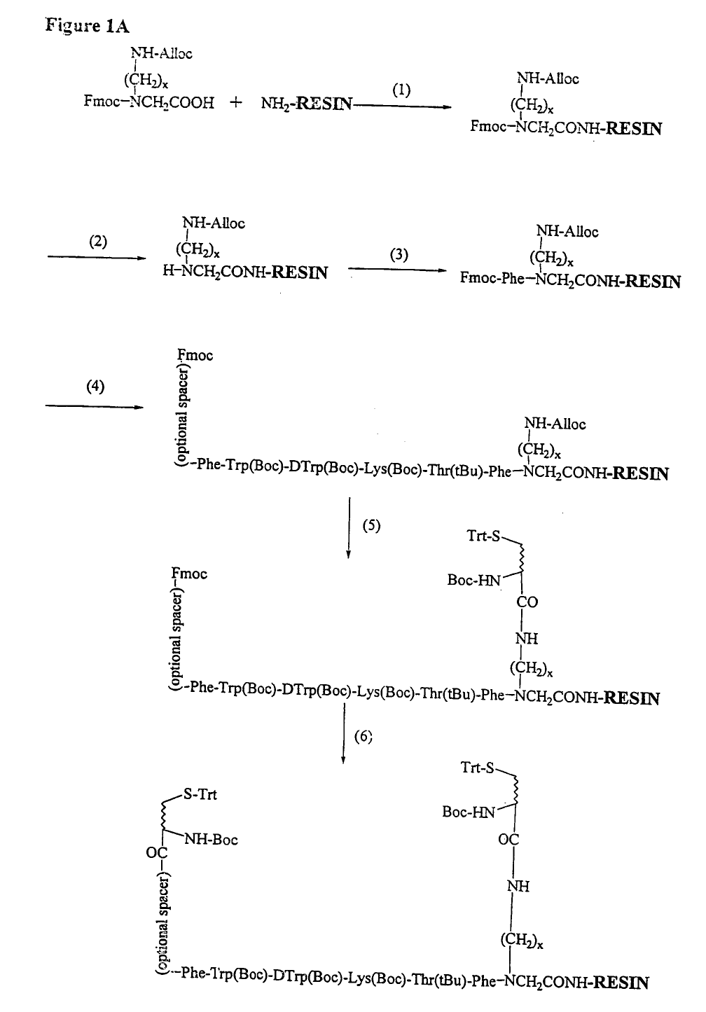

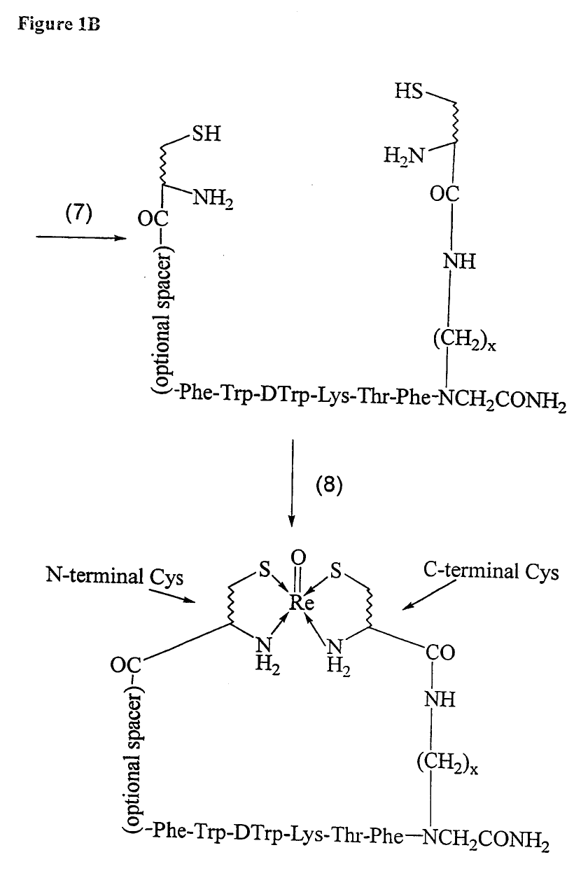

The synthesis scheme of the set of 48 peptides synthesized according to the general method above is described in FIG. 1. The compounds were backbone cyclized through site-specific complexation with ReO as crude peptides (example 2) and then purified.

Rhenium was chosen as the metal of choice as it is an excellent model for the radioactive isotopes ¹⁸⁶Re, ¹⁸⁸Re, and ⁹⁹Tc, which are most appropriate for medical applications due to the nature of their associated radiation and their half-life properties.

The selection of the metal atom for coordination, led us to the design of its binding site. Hence Re and Tc show the same preference for donor atoms S>N>>O. Re and Tc also prefer the same coordination geometry when they are in the +5 oxidation state. That is they adopt a square pyramidal structure, where 4 donor atoms are located in the square corners and a mono-oxo group is located above or below the square plane (with the metal located in the center of the pyramid). Because of the need for 4 donor atoms and because sulfur and nitrogen are the best donors, the Re binding site comprised two cysteines, one linked to the C-terminus Gly building unit nitrogen, and one to the last residue at the N-terminus each connected through the Cys carboxy group, to achieve on each side of the peptide a free thiol and a free amine for coordination with the Re atom.

### Example 2

### Reaction of Crude Metal-Free Peptides with Rhenium to Yield the oxorhenium(V) Complex

Crude peptide is dissolved in water and trichlorooxobis(triphenylphosphine)-rhenium(V) is added in DMF and the mixture is shaken at room temperature for about 2 hours. Removal of DMF is achieved by vacuum centrifugation (sample at 40° C.) for about 10 hours and the resulting product is purified by HPLC, yielding the oxorhenium(V) complex of the peptide.

### Example 3

### Design and Synthesis of 48 SST Peptide Analogs Backbone Cyclized through Metal Complexation

The compound denoted PTLR 3173 is a backbone cyclized somatostatin analog selective for SST-R2 and SST-R5. Its synthesis and activity are described in WO 99/65508.

The compound has the following structure: *GABA-Phe-Trp-DTrp-Lys-Thr-Phe-GlyC3*-NH₂ (wherein the asterisks indicate the cyclization points, SEQ ID NO: 2).

A set of 4S peptide analogs of PTR 3173 were synthesized according to the following formula:

Cys¹-Spacer-Phe-Trp-DTrp-Lys-Thr-Phe-GlyNX(Cys²)—NH2

wherein four parameters were varied systematically:

1) length of the methylene chain (X=2,3,6) on the Gly building unit linked to (Cys²);
2) configuration (L or D isomer) of the Cysteine residue (Cys²) linked to the 0)-amine of the Gly-building unit at the C-terminus;
3) a spacer (GABA, P alanine, Gly, or none), which connects the Cysteine residue at the N-terminus (Cys¹) to the peptide; and
4) configuration (L or D isomer) of the Cysteine residue (Cys¹) at the N-terminus.
   As following schemes describe the linear and cyclic structures of the resulted analogs: Linear structure:

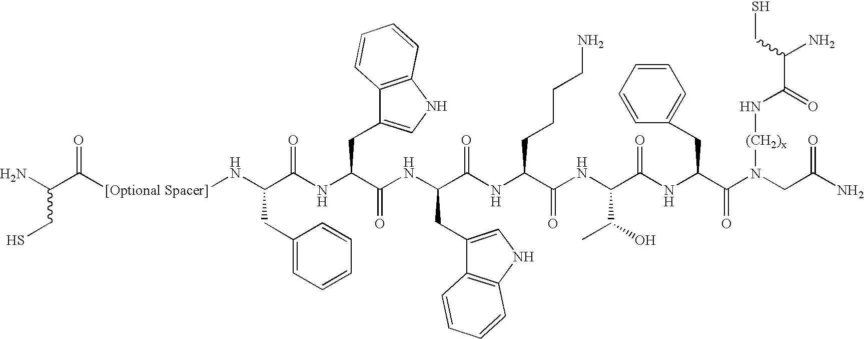

Backbone cyclized through metal complexation structure:

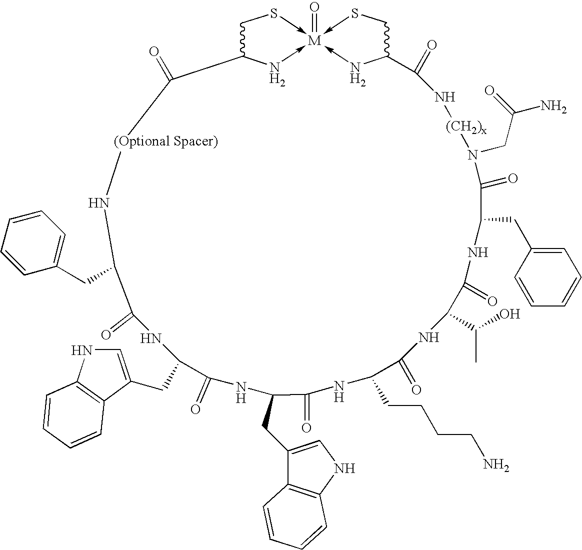

These variations led to a library of 48 peptides with different ring sizes (29 to 38 atoms), while in all members of the library the pharmacophore of PTR-3173 is reserved. Conformational diversity was expected within groups having the same ring size due to the differences in Cys configurations at the Re binding site and to different compositions (length of building unit chain and residue before Cys at the N-terminus). The compounds are described in Table No. 2:

TABLE NO 2

| ReO | Cys¹ | | X in | Cys² | Atoms in smallest |
|---|---|---|---|---|---|
| -GF- | isomer | Spacer | GlyNX | isomer | possible ring |
| 1 | L | GABA | 6 | L | 38 |
| 2 | L | GABA | 6 | D | 38 |
| 3 | D | GABA | 6 | L | 38 |
| 4 | D | GABA | 6 | D | 38 |
| 5 | L | β Alanine | 6 | L | 37 |
| 6 | L | β Alanine | 6 | D | 37 |
| 7 | D | β Alanine | 6 | L | 37 |
| 8 | D | β Alanine | 6 | D | 37 |
| 9 | L | Gly | 6 | L | 36 |
| 10 | L | Gly | 6 | D | 36 |
| 11 | D | Gly | 6 | L | 36 |
| 12 | D | Gly | 6 | D | 36 |
| 13 | L | none | 6 | L | 33 |
| 14 | L | none | 6 | D | 33 |
| 15 | D | none | 6 | L | 33 |
| 16 | D | none | 6 | D | 33 |
| 17 | L | GABA | 3 | L | 35 |
| 18 | L | GABA | 3 | D | 35 |
| 19 | D | GABA | 3 | L | 35 |
| 20 | D | GABA | 3 | D | 35 |
| 21 | L | β Alanine | 3 | L | 34 |
| 22 | L | β Alanine | 3 | D | 34 |
| 23 | D | β Alanine | 3 | L | 34 |
| 24 | D | β Alanine | 3 | D | 34 |
| 25 | L | Gly | 3 | L | 33 |
| 26 | L | Gly | 3 | D | 33 |
| 27 | D | Gly | 3 | L | 33 |
| 28 | D | Gly | 3 | D | 33 |
| 29 | L | none | 3 | L | 30 |
| 30 | L | none | 3 | D | 30 |
| 31 | D | none | 3 | L | 30 |
| 32 | D | none | 3 | D | 30 |
| 33 | L | GABA | 2 | L | 34 |
| 34 | L | GABA | 2 | D | 34 |
| 35 | D | GABA | 2 | L | 34 |
| 36 | D | GABA | 2 | D | 34 |
| 37 | L | β Alanine | 2 | L | 33 |
| 38 | L | β Alanine | 2 | D | 33 |
| 39 | D | β Alanine | 2 | L | 33 |
| 40 | D | β Alanine | 2 | D | 33 |
| 41 | L | Gly | 2 | L | 32 |
| 42 | L | Gly | 2 | D | 32 |
| 43 | D | Gly | 2 | L | 32 |
| 44 | D | Gly | 2 | D | 32 |
| 45 | L | none | 2 | L | 29 |
| 46 | L | none | 2 | D | 29 |
| 47 | D | none | 2 | L | 29 |
| 48 | D | none | 2 | D | 29 |

### Example 4

### Binding of Analogs to Somatostatin Receptors

The ability of the SST analogs of the invention to bind to SST receptors in vitro was demonstrated by assaying the ability of such analogs to inhibit binding of a radiolabelled SST analog to SST receptor-containing cell membranes as described above. The receptor membrane preparations used for these tests were from the cloned human receptors selectively and stably expressed in CHO cells and the radiolabelled analog used was (3([¹²⁵I]tyrosyl¹¹)SRIF-14.

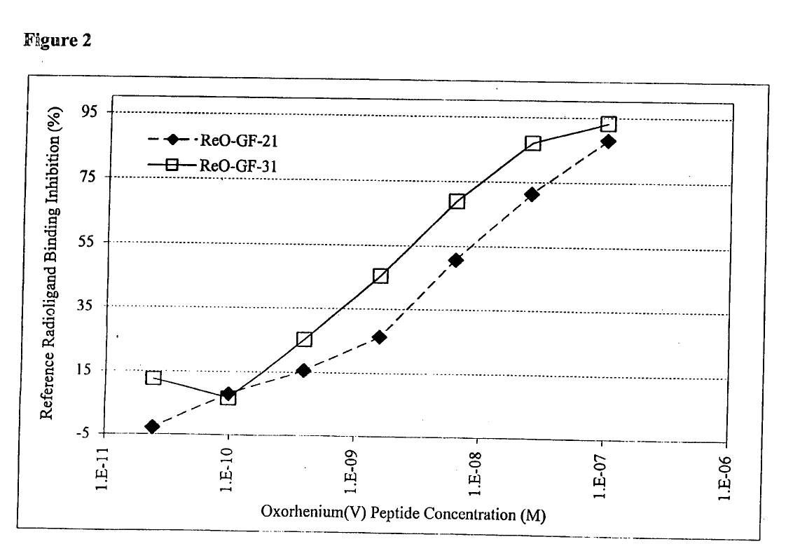

Table No. 3 describes the results of the binding assays of the 48 ReO-GF analogs to man cloned SST-R2 while FIG. 2 describes the competitive binding curves of the unds ReO-GF-21 and ReO-GF-31.

TABLE NO 3

| | | | | | Atoms in | |
|---|---|---|---|---|---|---|
| | | | | | smallest | |
| ReO | Cys¹ | | X in | Cys² | possible | IC_{50 nM} |
| -GF- | isomer | Spacer | GlyNX | isomer | ring | hSST-R2 |
| 1 | L | GABA | 6 | L | 38 | 16 |
| 2 | L | GABA | 6 | D | 38 | 3.8 |
| 3 | D | GABA | 6 | L | 38 | 6 |
| 4 | D | GABA | 6 | D | 38 | 12 |
| 5 | L | β Alanine | 6 | L | 37 | 35 |
| 6 | L | β Alanine | 6 | D | 37 | 5.3 |
| 7 | D | β Alanine | 6 | L | 37 | 12 |
| 8 | D | β Alanine | 6 | D | 37 | 5 |
| 9 | L | Gly | 6 | L | 36 | ˜10 |
| 10 | L | Gly | 6 | D | 36 | 3 |
| 11 | D | Gly | 6 | L | 36 | 4 |
| 12 | D | Gly | 6 | D | 36 | 9 |
| 13 | L | none | 6 | L | 33 | ˜10 |
| 14 | L | none | 6 | D | 33 | 7 |
| 15 | D | none | 6 | L | 33 | >10 |
| 16 | D | none | 6 | D | 33 | 18 |
| 17 | L | GABA | 3 | L | 35 | 24 |
| 18 | L | GABA | 3 | D | 35 | ˜10 |
| 19 | D | GABA | 3 | L | 35 | >10 |
| 20 | D | GABA | 3 | D | 35 | ˜10 |
| 21 | L | β Alanine | 3 | L | 34 | 1 |
| 22 | L | β Alanine | 3 | D | 34 | 11 |
| 23 | D | β Alanine | 3 | L | 34 | ˜10 |
| 24 | D | β Alanine | 3 | D | 34 | 23 |
| 25 | L | Gly | 3 | L | 33 | >10 |
| 26 | L | Gly | 3 | D | 33 | ˜10 |
| 27 | D | Gly | 3 | L | 33 | 20 |
| 28 | D | Gly | 3 | D | 33 | >10 |
| 29 | L | none | 3 | L | 30 | 2 |
| 30 | L | none | 3 | D | 30 | 13 |
| 31 | D | none | 3 | L | 30 | 1.6 |
| 32 | D | none | 3 | D | 30 | 14 |
| 33 | L | GABA | 2 | L | 34 | >10 |
| 34 | L | GABA | 2 | D | 34 | ˜100 |
| 35 | D | GABA | 2 | L | 34 | >10 |
| 36 | D | GABA | 2 | D | 34 | >10 |
| 37 | L | β Alanine | 2 | L | 33 | 3 |
| 38 | L | β Alanine | 2 | D | 33 | >10 |
| 39 | D | β Alanine | 2 | L | 33 | 10 |
| 40 | D | β Alanine | 2 | D | 33 | >10 |
| 41 | L | Gly | 2 | L | 32 | >10 |
| 42 | L | Gly | 2 | D | 32 | >10 |
| 43 | D | Gly | 2 | L | 32 | >10 |
| 44 | D | Gly | 2 | D | 32 | >10 |
| 45 | L | none | 2 | L | 29 | >10 |
| 46 | L | none | 2 | D | 29 | >10 |
| 47 | D | none | 2 | L | 29 | >10 |
| 48 | D | none | 2 | D | 29 | >10 |

Following oxorhenium(V) complexation of the crude linear peptides to provide the cyclic-through-metal complexes, semi-preparative HPLC purification was performed. Multiple HPLC fractions were collected as the desired product eluted, resulting in more than one fraction containing the correct mass (according to MS). Since up to four conformers (cis-endo, cis-exo, trans-endo, trans-exo) were predicted for each of the 48 peptide complexes, multiple HPLC peaks were expected and observed in the crude analytical chromatograms of the complexation reaction and in the semi-preparative chromatograms. The HPLC fractions presumably contained mixtures of isomers as occasionally more than one peak was observed. Screening was performed on the fractions without further purification by estimating the concentration of peptide against a PTR 3173 standard. Thus for each peptide, more than one fraction was analyzed and each fraction presumably contained a different distribution of conformers.

The data presented in the above table show that the peptides of the instant invention have a high affinity of binding for human SST-R2. Selected most active analogs identified are summarized in the following table:

TABLE 4

| | | |
|---|---|---|
| Compound | | IC₅₀ |
| ReO-GF- | Sequence | (nM) |

| | | |
|---|---|---|
| 29 | ReO-LCys*-Phe-Trp-DTrp-Lys-Thr-Phe-GlyN3(LCys*)-NH₂ | 2 |
| 31 | ReO-DCys*-Phe-Trp-DTrp-Lys-Thr-Phe-GlyN3(LCys*)-NH₂ | 1.6 |
| 21 | ReO-LCys*-βAla-Phe-Trp-DTrp-Lys-Thr-Phe-GlyN3(LCys*)-NH₂ | 1 |
| 37 | ReO-LCys*-βAla-Phe-Trp-DTrp-Lys-Thr-Phe-GlyN2(LCys*)-NH₂ | 3 |
| 10 | ReO-LCys*-Gly-Phe-Trp-DTrp-Lys-Thr-Phe-GlyN6(DCys*)-NH₂ | 3 |
| 11 | ReO-DCys*-Gly-Phe-Trp-DTrp-Lys-Thr-Phe-GlyN6(LCys*)-NH₂ | 4 |
| 06 | ReO-LCys*-βAla-Phe-Trp-DTrp-Lys-Thr-Phe-GlyN6(DCys*)-NH₂ | 5.3 |
| 03 | ReO-DCys*-GABA-Phe-Trp-DTrp-Lys-Thr-Phe-GlyN6(LCys*)-NH₂ | 6 |
| 08 | ReO-DCys*-βAla-Phe-Trp-DTrp-Lys-Thr-Phe-GlyN6(DCys*)-NH₂ | 5 |
| 14 | ReO-LCys*-Phe-Trp-DTrp-Lys-Thr-Phe-GlyN6(DCys*)-NH₂ | 7 |
| 02 | ReO-LCys*-GABA-Phe-Trp-DTrp-Lys-Thr-Phe-GlyN6(DCys*)-NH₂ | 3.8 |
| 12 | ReO-DCys*-Gly-Phe-Trp-DTrp-Lys-Thr-Phe-GlyN6(DCys*)-NH₂ | 9 |

wherein the asterisks denote the chelating groups used for cyclization through metal complexation.

The properties of six compounds from the above table having IC₅₀<5 nM are characterized in Table no. 5:

TABLE NO 5

| ReO- | Cys¹ | | X in | Cys² | Atoms in smallest |
|---|---|---|---|---|---|
| GF- | isomer | Spacer | GlyNX | isomer | possible ring |
| 10 | D | Gly | 6 | L | 36 |
| 11 | L | Gly | 6 | D | 36 |
| 21 | L | ΔAla | 3 | L | 34 |
| 29 | L | none | 3 | L | 30 |
| 31 | L | none | 3 | D | 30 |
| 37 | L | ΔAla | 2 | L | 33 |

### Example 5

### Localization and In Vivo Imaging of SST-R—Expressing Tumors in Rats

In vivo imaging of SST receptors expressed by rat tumor cells is performed essentially as described by Bakker et al. (1991, Life Sciences 42:1593-1601). Tumor cells are implanted intramuscularly in a suspension of 0.05 to 0.1 mL/animal, into the right hind thigh of 6 week old rats. The tumors are allowed to grow to approximately 0.5 to 2 g, harvested, and tumor brei is used to implant a second, naive set of Lewis rats. Passaging in this fashion is repeated to generate no more than five successive generations of tumor-bearing animals. The tumor-bearing animals used for the in vivo studies are usually from the third to fifth passage and bearing 0.2 to 2 g tumors. For studies of the specificity of radiotracer localization in the tumors, selected animals are given an subcutaneous SST-R blocking dose (4 mg/kg) of Octreotide 30 minutes prior to injection of the radiotracer. (This protocol has been shown by Bakker et al. to result in a lowering of ¹¹¹In-DTPA-Gctreotide tumor uptake by 40%). Third-to fifth-passage tumor-bearing rats are injected intravenously via the dorsal tail vein with a dose of 0.15-0.20 mCi 99mTc-labeled compound corresponding to 3 to 8 μg peptide in 0.2 to 0.4 mL. At selected times, the animals are sacrificed by cervical dislocation and harvested tissue samples are weighed and counted along with an aliquot of the injected dose in a gamma well-counter.

While the present invention has been described for certain preferred embodiments and examples it will be appreciated by the skilled artisan that many variations and modifications may be performed to optimize the activities of the peptides and analogs of the invention. The examples are to be construed as non-limitative and serve only for illustrative purposes of the principles disclosed according to the present invention, the scope of which is defined by the claims which follow.

1. A somatostatin analog of three to twenty-four amino acids that incorporates at least one building unit, comprising a N^{α}-ω-functionalized derivative of an amino acid, wherein a backbone cyclic structure is formed by metal complexation to a chelating moiety comprising the at least one building unit and a second moiety selected from the group consisting of a second building unit, the side chain of an amino acid residue of the sequence or a terminal amino acid residue.

2. The somatostatin analog of claim 1 wherein the chelating moiety comprises four donor atoms.

3. The somatostatin analog of claim 2 wherein the chelating moiety is N₂S₂ type.

4. The somatostatin analog of claim 1 comprising an analog having the general Formula No. 3 (SEQ ID NO: 1): 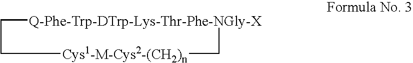
wherein n is 1 to 6; Q is absent or is selected from the group consisting of gamma amino butyric acid (GABA), Gly, and βAla; X designates a terminal carboxy acid, amide or alcohol group; Cys¹ and Cys² are each independently L or D isomers; and M is a metal.

5. The somatostatin analog of claim 4 wherein: n is 2,3, or 6; Q is absent or is βAla; Cys² IS LCys; X is an amide; and M is a radiometal selected from the group consisting of [^{nat}Re] oxorhenium (V), [¹⁸⁶Re] oxorhenium (V), [¹⁸⁸Re] oxorhenium (V) or [^{99m}Tc] oxotechnetium (V).

6. The sonratostatin afialog of claim 4 selected from the group consisting of:
ReO-LCvs*-Phe-Trp-DTrp-Lys-Thr-Phe-GlyN3 (LCys*)-NH2;
ReO-DCys*-Phe-Trp-DTrp-Lys-Thr-Phe-GlyN3 (LCys*)-NH2;
ReO-LCys*-βAla-Phe-Trp-DTrp-Lys-Thr-Phe-GlyN3 (LCys*)-NH2;
ReO-LCys*-βAla-Phe-Trp-DTrp-Lys-Thr-Phe-GlyN2 (LCys*)-NH2;
ReO-LCys*-Gly-Phe-Trp-DTrp-Lys-Thr-Phe-GlyN6 (DCys*)-NH2;
ReO-DCys*-Gly-Phe-Trp-DTrp-Lys-Thr-Phe-GlyN6 (DCys*)-NH2;
ReO-LCys*-βAla-Phe-Trp-DTrp-Lys-Thr-Phe-GlyN6 (DCys*)-NH2;
ReO-DCys*-GABA-Phe-Trp-DTrp-Lys-Thr-Phe-GlyN6 (DCys*)-NH2;
ReO-DCys*-βAla-Phe-Trp-DTrp-Lys-Thr-Phe-GlyN6 (DCys*)-NH2;
ReO-LCys*-Phe-Trp-DTrp-Lys-Thr-Phe-GlyN6 (DCys*)-NH2;
ReO-LCys*-GABA-Phe-Trp-DTrp-Lys-Thr-Phe-GlyN6 (DCys*)-NH2;
ReO-DCys*-Gly-Phe-Trp-DTrp-Lys-Thr-Phe-GlyN6 (DCys*)-NH2;
wherein the asterisks denote the chelating groups used for cyclization through metal complexation.

7. The somatostatin analog of claim 1 wherein the chelating moiety comprises a complek with a radioisotope.

8. The somatostatin analog according to claim 7 wherein the radioisotope is selected from 99 mTc, 186Re and 188Re.

9. A radiolabelled peptide analog comprising a somatostatin analog of claim 1 wherein the backbone cyclic structure is formed by complexation of a radioactive metal to the chelating moiety.

10. A pharmaceutical composition comprising a somatostatin analog of claim 1 and a pharmaceutically acceptable carrier.

11. A method for diagnosing or treating cancer or allograft rejections in a mammal which comprises administering to a mammal in need of such diagnosis or treatment a somatostatin analog according to claim 1 in an amount effective to assist in the diagnosis or treatment of the mammal.

12. The method according to claim 11 wherein the somatostatin analog is administered in a pharmaceutical composition that includes the analog and a pharmaceutically acceptable carrier.

13. The method according to claim 11 which further comprises imaging metastases with the somatostatin analog.

14. The method according to claim 11 which further comprises labeling the somatostatin analog with a detectable tracer.

15. The method according to claim 11 which further comprises administering a somatostatin analog that is selective for one somatostatin receptor subtype.

16. The method according to claim 11 which further comprises administering a somatostatin analog that is selective for two or more somatostatin receptor subtypes.

17. A method for treating disorders selected from the group consisting of cancers, autoimmune diseases, endocrine disorders, diabetes-associated complications, gastrointestinal disorders, inflammatory diseases, pancreatitis, atherosclerosis, restenosis, allograft rejection, and post-surgical pain, which comprises administering to a mammal in need thereof a therapeutically effective amount of the somatostatin analog according to claim 1.

18. A method for diagnosing disorders selected from the group consisting of cancers, autoimmune diseases, endocrine disorders, diabetes-associated complications, gastrointestinal disorders, inflammatory diseases, pancreatitis, atherosclerosis, restenosis, allograft rejection, and post-surgical pain, which comprises administering to a mammal in need thereof a diagnosis effective amount of the somatostatin analog according to claim 1.

19. A kit for preparing a scintigraphic imaging agent for imaging sites within a mammalian body, said kit comprising a somatostatin analog backbone cyclized through metal complexation.

20. A Method for scintigraphic imaging of sites within a mammalian body which comprises preparing a reagent by reacting a somatostatin analog according to claim 1 with a radiometal and appropriate additive for reduction of the metal, to form a backbone cyclic radiolabelled peptide; and utilizing the reagent for scintigraphic imaging.


SEQUENCE LISTING (2 sequences)

SEQ ID NO: 1 - protein, 9 aa, Artificial sequence
  synthetic peptide
CXFWWKTFX

SEQ ID NO: 2 - protein, 8 aa, Artificial sequence
  synthetic peptide
XFWWKTFX